(19) United States
(12) Patent Application Publication
(10) Pub. No.: US 2014/0194373 A1
(43) Pub. Date: Jul. 10, 2014

(54) COSMETIC USE OF HESPERIDIN OR A DERIVATIVE THEREOF IN THE PREVENTION AND/OR TREATMENT OF BODY ODOUR

(71) Applicants: NESTEC S.A., Vevey (CH); L'OREAL, Paris (FR)

(72) Inventors: Isabelle Castiel, Nice (FR); Audrey Gueniche, Rueil Malmaison (FR)

(21) Appl. No.: 14/240,278

(22) PCT Filed: Sep. 27, 2012
(86) PCT No.: PCT/IB2012/055144

§ 371 Date: Feb. 21, 2014

Related U.S. Application Data
(60) Provisional application No. 61/543,006, filed on Oct. 4, 2011.

(30) Foreign Application Priority Data
Sep. 27, 2011 (FR) 1158620

(51) Int. Cl. A61K 31/7048; A23L 1/30; A61K 9/00

(52) U.S. Cl. CPC A61K 31/7048; A61K 9/0053; A23L 1/3002
USPC 514/27

(57) ABSTRACT

The present invention relates to the cosmetic use, in particular the oral use, of at least an effective amount of hesperidin or a derivative thereof as a deodorant agent.

The present invention relates to the field of cosmetic and/or dermatological products, more particularly intended for combating body odour.

In particular, the present invention is directed towards proposing the use of a novel active agent for preventing and/or treating body odour.

The invention relates especially to the oral use of hesperidin or a derivative thereof as an active agent for treating and/or preventing body odour.

The invention relates more particularly to the cosmetic use, especially orally, of hesperidin or a derivative thereof as a deodorant agent.

Perspiration, or sweating, is the removal of sweat via the skin pores. This physiological function is present to varying degrees in all mammals. In man, its main function is to regulate the temperature and to remove toxins.

Sweat is a liquid secreted by two types of sweat gland.

The eccrine glands are located over the entire body surface. Eccrine sweat is transparent, aqueous and odourless. It plays a role in moisturizing the horny layer and in microbiological protection, and also in gripping and in percutaneous absorption.

The apocrine glands are more particularly located in the armpits, the auditory canals, the mammary areolae and the perianal regions. The excretory part is located at the top of the hair follicle. These glands do not become functional until puberty. Apocrine sweat is viscous, of milky appearance and slightly odorous. It is rich in lipids and also contains proteins. This secretion is regulated psychically.

Sweat is sterile and odourless or only sparingly odorous on secretion.

Body odour is caused by the bacterial degradation of apocrine sweat. Moisture, sebaceous secretions, pilosity, hygiene and the diet are also responsible for body odour.

The underarm region is the area of the body where the most bacteria are found. These bacteria produce volatile and malodorous compounds.

The underarm regions contain a large range of bacteria (about 10⁶ cells/cm²), most of which are Gram-positive bacteria. These bacteria include the following species: Staphylococcus, Micrococcus, anaerobic/microaerobic Propionibacteria, and aerobic coryneform bacteria, which themselves consist of the species Corynebacterium (83%), Brevibacterium (5%) and other coryneforms (12%).

Studies have shown that the majority of the underarm coryneforms are comparable either to Corynebacterium G-2 CDC G5840 or to Corynebacterium mucifaciens DMMZ 2278.

There is a correlation between the composition of corynebacteria and the intensity of the odour formed. Thus, people with poor underarm odour generally have a flora composition different from people who do not have poor underarm odour.

It is thus the contact of sweat and more particularly of apocrine sweat with the bacteria mentioned above which induces the formation of volatile and malodorous compounds, via the bacterial degradation of this sweat.

These compounds derived from the degradation of sweat, which are the cause of the unpleasant odour, are especially represented by C₂-C₅ short-chain volatile acids, malodorous steroids, such as the steroid 16-androstene, C₆-C₁₂ fatty acids, such as the trans (E) isomer of 3-methyl-2-hexenoic acid (3M2H), or sulfanylalkanols.

Finally, pH 6 is the optimum pH as regards the production of odour stimulated by coryneforms. There is no formation of unpleasant odour at pH 4 or at pH 8. Studies have shown that the pH of the underarm skin is 5.9 in the morning and becomes 5.5 in the evening. These two values concur with the pH value at which unpleasant odour is formed.

Body odour is a biological phenomenon that is important to control efficiently.

To combat perspiration and unpleasant odour, various compounds have already been proposed, which, by topical application to the skin, are capable of inhibiting sweating, of inhibiting the bacteria responsible for the chemical transformations, of masking odours and/or of reducing and/or destroying the indigenous microflora.

To act on unpleasant odours it has generally been proposed:
to act on the sweat glands by blocking the process of perspiration or at least by regulating it;
to act on the degradation of sweat by using enzymatic or non-enzymatic bactericides; and/or
to act on the emission of odour with the aid of fragrances or odour absorbers.

The bactericide most commonly used is Triclosan (2,4,4-trichloro-2-hydroxy-diphenyl ether). However, it has the drawback of modifying all the cutaneous micro flora and of being inhibited by certain compounds, often used in cosmetics.

It is also possible to block volatile fatty acids by adding, to the areas of perspiration, compounds that are capable of trapping the volatile fatty acids or of raising the pH to prevent the formation thereof and to prevent the proliferation of the bacterial flora.

It is also possible to act on sweat by decreasing or regulating perspiration. To do this, use may be made of active substances of antiperspirant type which have the effect of limiting the flow of sweat produced. However, they generally contain as active principles aluminium salts, which prove to be irritant to the skin and which decrease the flow of sweat by modifying the skin physiology.

Among the other denaturing substances used, glutaraldehyde and formaldehyde are also known.

A more extreme solution consists of a surgical operation such as sympathectomy, excision, curettage or liposuction of the areas that are the most active in the secretion of sweat. However, there is a risk of appearance of compensatory phenomena such as the formation of cheloids, pneumothorax or hyperhydrosis.

Finally, sweating may be treated medicinally or by injection of toxins. Tranquilizers, sedatives/spasmolytic agents and anticholinergic agents are suitable for use, although the latter often entail a very large number of side effects.

There thus remains a need for novel active agents that are capable of exerting a cosmetic or therapeutic action on body odour.

There is also a need for active agents for re-establishing the ecoflora of the armpits, the auditory canals, the mammary areolae and the perianal regions where the majority of the apocrine sweat glands are found.

There is also a need to be able to act simply on all these areas, or even to act globally on the skin as a whole, or even on its deep layers.

There is also a need to degrade, inhibit and/or modify the bacteria present in the underarm regions, the perianal areas, the feet and the mammary areolae, which are involved in the degradation of sweat and/or sebum, more particularly apocrine sweat.

The object of the present invention is to satisfy these needs.

One subject of the present invention is thus the cosmetic use, especially orally, of at least an effective amount of hesperidin or a derivative thereof as a deodorant agent.

A subject of the invention is also the cosmetic use of at least an effective amount of hesperidin or a derivative thereof as an active agent for stimulating the expression of dermicidin in the epidermal stratum corneum.

The reason for this is that the inventors have found that hesperidin, or a derivative thereof, proves to be capable of stimulating the expression of dermicidin in the epidermis, as demonstrated by the results presented in the present text.

Hesperidin, or a derivative thereof, may thus especially be used in a composition intended for oral, parenteral or topical application.

The topical route makes it possible to deploy all the properties of the active agents at a precise site, on the areas to be treated.

The oral route has the significant advantage of acting more globally on the skin as a whole and in its deep layers.

In the present invention, the oral route is preferred.

For the purposes of the present invention, the terms “prevent” or “prevention” mean reducing the risk of occurrence of the phenomenon concerned, and the term “effective amount” denotes an amount that is sufficient to obtain the expected effect.

For the purposes of the present invention, the term “body odour” is intended to denote any undesirable odour that may be perceived as unpleasant, or even incapacitating, by the individual producing it and/or those in his vicinity.

The body odour more particularly considered according to the invention is that associated directly or indirectly with the presence of bodily fluids secreted on an epidermis, such as sweat and sebum, especially those arising from the degradation and/or from the secretion of sweat or of sebum, and more particularly those arising from the bacterial degradation of sweat, and especially of apocrine sweat.

The odour(s) concerned are especially those noted on areas of the body that are subject to heavy sweating and more particularly the underarm regions, especially the armpits, or else the perianal regions, the feet and the mammary areolae.

Hesperidin or a derivative thereof, and also a composition according to the invention comprising the same, is thus more particularly used for degrading, inhibiting and/or modifying the bacteria present in the underarm regions, the perianal areas, the feet and the mammary areolae, and involved in the degradation of sweat and/or sebum, more particularly apocrine sweat.

To the inventors' knowledge, the activity of hesperidin or a derivative thereof orally as a deodorant agent has never been described.

Admittedly, hesperidin is already proposed for cosmetic or even therapeutic active-agent purposes. However, hesperidin is known essentially for the following types of action:

1) anti-ageing, antioxidant, UV-related prevention;

2) on skin beauty by regulating sebum, increasing cell proliferation, cell regeneration and repair, improving the structure of the skin, and also for its action on hair growth and the prevention of alopecia;

3) antiinflammatory, antiallergic; and

4) skin lightening.

For its part, document EP 1 430 879 teaches the topical use of flavonoids, and more particularly of hesperidin, as arylsulfatase inhibitors.

As emerges from the text hereinbelow, the inventors have especially found that hesperidin shows stimulatory activity on the expression of a major protein involved in the antimicrobial defence of the epidermis, dermicidin, which is capable of promoting and reinforcing the epidermal defences against the excessive colonization of the epidermis.

This stimulation has the advantage of efficiently combating colonization of the epidermis by microorganisms, and especially by the Corynebacteria responsible for unpleasant odours. This decrease obtained using hesperidin or a derivative thereof thus contributes towards re-establishing an equilibrated ecoflora with, as a consequence, a reduction in body odour.

The invention also relates to the use of hesperidin or a derivative thereof in which the hesperidin or a derivative thereof is used in an oral composition for treating and/or preventing body odour.

Thus, according to one particular embodiment, a subject of the invention is the cosmetic use of a composition comprising an effective amount of hesperidin or a derivative thereof for treating and/or preventing body odour, via the oral route.

According to another of its aspects, a subject of the present invention is also a process, especially a cosmetic process, for preventing and/or treating body odour, comprising at least one step of oral administration of hesperidin or a derivative thereof.

According to one embodiment, the hesperidin, or a derivative thereof, used in the process of the invention may be present in a composition in accordance with the invention.

According to one embodiment, such a composition may be administered to an individual.

As specified hereinbelow, the compositions in accordance with the invention are formulated so as to be compatible with the selected mode of administration.

Hesperidin

Hesperidin belongs to the family of flavanones, which are natural glucoside compounds found mainly in citrus fruit, i.e. fruit of the genus Citrus, for instance oranges, lemons or bitter oranges, or alternatively grapes.

They are predominantly present in citrus peel, but are also found in large amounts in the pulp, and thus in the juice of citrus fruit.

Hesperidin is a glycosyl compound comprising a hesperitin flavanone nucleus (3′,5′,5-trihydroxy-4′-methoxyflavanone) to which is covalently bonded a glucoside part of rutinose (L-rhamnosyl-(α1→6)-glucose) attached to the hydroxyl group present on the carbon in position 7 of hesperitin.

The term “hesperidin” thus means the compound (S)-7-[[6-0-(6-deoxy-α-L-mannopyranosyl)-β-D-glucopyranosyl]oxy]-2,3-dihydro-5-hydroxy-2-(3-hydroxy-4-methoxyphenyl)-4H-1-benzopyran-4-one.

The hesperidin derivatives may be chosen from the aglycone forms thereof, the chalcone forms thereof, the glycosyl forms thereof and the methylated forms thereof, and also the sulphate or glucuronide forms thereof, which are found as metabolic products in the blood circulation.

The hesperidin derivatives may be obtained by various processes known to those skilled in the art, for instance enzymatic treatments, or alternatively may be obtained by synthesis. Glucose-7-hesperitin may thus be prepared via a treatment with rhamnosidase or hesperidinase.

Hesperidin derivatives that may especially be mentioned include the following compounds:
the compound hesperitin, consisting of a non-glycosyl flavanone nucleus of hesperidin, which has the following formula: (S)-2,3-dihydro-5,7-dihydroxy-2-(3-hydroxy-4-methoxyphenyl)-4H-1-benzopyran-4-one; 3′,5,7-trihydroxy-4′-methoxyflavanone;
α-glucosyl-hesperidin, which comprises a chain of 1 to 20 glucose residues bonded together via a 1,4 bond, the chain of glucose residues itself being bonded via bonding of 1,4 type in position 4 of the glucose residue of hesperidin; these hesperidin derivatives and the process for preparing them are described especially in patent application EP 0 825 196 and in U.S. Pat. No. 6,048,712;
methyl-hesperidin compounds, especially the compound 3¹-methyl-7-(rhamnosyl-2-methylglucosyl)hesperidin and the compound 3¹-methylhesperidin, and
conjugates of hesperitin and of sulfate or glucuronide, which are found, with hesperitin, as products of metabolization of hesperidin in the blood circulation,
neohesperidin dihydrochalcone.

According to one particular embodiment, hesperidin and derivatives thereof more preferentially chosen from hesperitin and hesperitin glucuronide are used.

In general, the effective amount of hesperidin or derivative thereof may represent a content of between 0.001% and 30% by weight and preferably between 0.01% and 20% by weight relative to the total weight of a composition containing the same.

Via the oral route, the daily dose of hesperidin or derivative thereof may advantageously be from 0.5 to 2500 mg per day and preferably from 5 to 500 mg per day.

Active Agent

Hesperidin may advantageously be combined with at least one other active agent.

Thus, a topical or oral, preferably oral, composition according to the invention may also advantageously contain at least one anti-sweat active agent.

Such a formulation makes it possible advantageously to amplify the beneficial effects of the hesperidin of the invention or of a derivative thereof.

A composition according to the invention may also contain at least one additional active agent for preventing and/or treating body odour, other than hesperidin or a derivative thereof.

An additional active agent for preventing and/or treating body odour, other than hesperidin or a derivative thereof, which is suitable for use in the invention may be chosen especially from active agents for regulating and/or inhibiting sweating, for instance an anti-sweat or antiperspirant active agent.

Such agents may especially be suitable for:
reducing and/or inhibiting the activity of the apocrine and/or eccrine glands, especially the apocrine glands, or reducing and/or inhibiting the flow of sweat produced. Such agents may be chosen, for example, from aluminium and/or zirconium salts, glutaraldehyde and formaldehyde;
reducing and/or inhibiting the degradation of sweat, especially of apocrine sweat, by the bacteria that are the cause of body odour. Such agents may be, for example, enzymatic or non-enzymatic bactericides, and more particularly triclosan or farnesol;
inhibiting the enzymes responsible for the development of odour;
limiting the growth of bacteria. Such agents may be, for example, transition metal-chelating agents such as EDTA or DPTA;
preventing the formation of and/or inhibiting and/or destroying the compounds derived from degradation of sweat which are responsible for body odour; such agents may be, for example, C₂-C₅ short-chain volatile fatty acids, malodorous steroids (16-androstene steroids), C₆-C₁₂ acids, or sulfanylalkanols. Such active agents may also be chosen from arylsulfatase, 5-lipoxygenase, aminocylase or B-glucuronidase inhibitors; and/or
acting on the odour per se, for instance fragrances or odour absorbers, for instance zeolites, cyclodextrins, metal oxide silicates, especially those described in US 2005/063 928, metal oxide particles modified with a transition metal, especially those described in US 2005/084 464 and US 2005/084 474, aluminosilicates, especially those described in EP 1 658 863, or nanometric chitosan derivative particles, especially those described in U.S. Pat. No. 6,916,465.

Active agents that block volatile fatty acids or that raise the pH in order to prevent the formation of the volatile fatty acids and to prevent the proliferation of the bacterial flora may also be combined with a composition in accordance with the invention.

The additional active agent is present, for example, in a content ranging from 0.1% to 10% by weight, preferentially from 0.1% to 5% by weight and preferably from 0.5% to 3% by weight relative to the total weight of the composition comprising it.

Besides these active agents, the compositions according to the invention may also contain several other active agents that are commonly used and/or permitted.

As active agents that are conventionally used, mention may be made of vitamins B3, B5, B6, B8, C, D, E or PP, niacin, carotenoids, polyphenols, minerals and trace elements, phytoestrogens, proteins and amino acids, mono- and polysaccharides, amino sugars, phytosterols and triterpene alcohols of plant origin.

The minerals and trace elements that may particularly be used are zinc, calcium, magnesium, copper, iron, iodine, manganese, selenium and chromium (III).

Among the polyphenols, use may be made in particular of polyphenols from grape, tea, olive, cocoa, coffee, apple, blueberry, elderberry, strawberry, cranberry and onion.

Preferably, among the phytoestrogens that may be used according to the invention, the ones that are selected are isoflavones in free or glycosyl form, such as genistein, daidzein, glycitein or lignans, in particular those from flax and from Schizandra chinensis.

Amino acids or peptides and proteins containing them, such as taurine, threonine, cysteine, tryptophan and methionine, may also be used in the present invention.

The lipids in accordance with the invention preferably belong to the group of oils containing mono- and polyunsaturated fatty acids such as oleic acid, linoleic acid, α-linolenic acid, γ-linolenic acid or stearidonic acid, long-chain fish omega-3 fatty acids, such as EPA and DHA, or alternatively conjugated fatty acids derived from plants or animals, such as CLA (conjugated linoleic acid), monounsaturated fatty acids such as petroselinic acid or any extract containing the same, such as coriander oil.

In particular, an antioxidant complex comprising vitamins C and E and at least one carotenoid, especially a carotenoid chosen from β-carotene, lycopene, astaxanthin, zeaxanthin and lutein, flavonoids other than hesperidin or a derivative thereof, such as catechins, proanthocyanidins and anthocyanins, lipoic acid and coenzyme Q10, may be used.

A related active agent that may be suitable for use in the invention may also be a prebiotic agent or a mixture of prebiotic agents. More particularly, these prebiotic agents may be chosen from oligosaccharides produced from glucose, galactose, xylose, maltose, sucrose, lactose, starch, xylan, hemicellulose, inulin, and gums of acacia type, for example, or mixtures thereof.

In particular, the oligosaccharide comprises at least one fructo-oligosaccharide. More particularly, this prebiotic agent may comprise a mixture of fructo-oligosaccharide and insulin.

In the topical galenical forms, use may be made more particularly, as hydrophilic active agents of proteins or protein hydrolysates, amino acids, polyols, especially C₂ to C₁₀ polyols such as glycerols, sorbitol, butylene glycol and polyethylene glycol, urea, allantoin, sugars and sugar derivatives, water-soluble vitamins, starch, and bacterial or plant extracts such as those from Aloe vera.

As regards the lipophilic active agents, use may be made of retinol (vitamin A) and derivatives thereof, tocopherol (vitamin E) and derivatives thereof, ceramides, essential oils or unsaponifiable materials (tocotrienol, sesamine, gamma-oryzanol, phytosterols, squalenes, waxes and terpenes).

A related active agent that may also be suitable for use in the invention may be a probiotic microorganism or a mixture of probiotic microorganisms.

These microorganisms that are suitable for use as related active agent according to the invention may be chosen especially from ascomycetes such as Saccharomyces, Yarrowia, Kluyveromyces, Torulaspora, Schizosaccharomyces, Debaromyces, Candida, Pichia, Aspergillus and Penicillium, bacteria of the genus Bifidobacterium, Bacteroides, Fusobacterium, Melissococcus, Propionibacterium, Enterococcus, Lactococcus, Staphylococcus, Streptococcus, Peptostrepococcus, Bacillus, Pediococcus, Micrococcus, Leuconostoc, Weissella, Aerococcus, Oenococcus and Lactobacillus and mixtures thereof.

As probiotic ascomycetes that are most particularly suitable for use in the present invention, mention may be made in particular of Yarrowia lipolitica and Kluyveromyces lactis, and also Saccharomyces cereviseae, Torulaspora spp., Schizosaccharamyces pombe, Candida spp. and Pichia spp.

Examples of specific probiotic bacteria are Bifidobacterium bifidum, Bifidobacterium infantis, Bifidobacterium longum, Lactobacillus acidophilus, Lactobacillus alimentarius, Lactobacillus casei subsp. Casei, Lactobacillus casei Shirota, Lactobacillus paracasei, Lactobacillus curvatus, Lactobacillus delbruckii subsp. Lactis, Lactobacillus gasseri, Lactobacillus johnsonii, Lactobacillus reuteri, Lactobacillus rhamnosus (Lactobacillus GG), Lactobacillus sake, Lactococcus lactis, Streptococcus thermophilus, Staphylococccus carnosus, and Staphylococcus xylosus, Propionibacterium freundereichii and mixtures thereof.

More particularly, they are probiotic microorganisms derived from the group of lactic acid bacteria, especially such as Lactobacilli and/or Bifidobacterium. As illustrations of these lactic acid bacteria, mention may be made more particularly of Lactobacillus johnsonii, Lactobacillus reuteri, Lactobacillus rhamnosus, Lactobacillus paracasei, Lactobacillus casei or in the genus Bifidobacterium species, the species including: Bifidobacterium longum, Bifidobacterium bifidum, Bifidobacterium breve, Bifidobacterium animalis, Bifidobacterium lactis, Bifidobacterium infantis, Bifidobacterium adolescentis or Bifidobacterium pseudocatenulatum and mixtures thereof.

In the strains of probiotic bacteria, the species that are particularly suitable for use are Lactobacillus johnsonii, Bifidobacterium adolescentis and Bifidobacterium longum, which are filed, respectively, according to the Treaty of Budapest at the Institut Pasteur (28 rue du Docteur Roux, F-75024 Paris cedex 15) under the following designations CNCM 1-1225, CNCM 1-2168 and CNCM 1-2170, and the genus Bifidobacterium lactis (Bb 12) (ATCC27536). The strain Bifidobacterium lactis (ATCC27536) may be obtained from Hansen (Chr. Hansen A/S, 10-12 Boege Alle, P.O. Box 407, DK-2970 Hoersholm, Denmark).

Active agents such as moisturizing active agents of reference in cosmetics, for instance glycerol, hyaluronic acid, urea and derivatives thereof, and also active agents that promote desquamation and peeling, such as chelating agents, jasmonic acid and derivatives thereof, in particular ER2412, reducing compounds, sulfonic derivatives and particularly HEPES, amino acids, AHAs and BHAs, in particular glycolic acid and ER195, and certain detergents, may also advantageously be combined with the product.

Galenical Forms

The compositions according to the invention may be in any galenical form normally available for the selected mode of administration.

The support may be of diverse nature according to the type of composition under consideration.

In the case of using a composition in accordance with the invention via the oral route, the use of an ingestible support is preferred.

The ingestible support may be of diverse nature according to the type of composition under consideration.

Food or pharmaceutical ingestible supports that are particularly suitable for use include milk, yogurt, cheese, fermented milks, milk-based fermented products, ice creams, products based on fermented cereals, milk-based powders, infant and baby formulas, food products of confectionary type, chocolates, cereals, animal feed in particular for pets, tablets, gel capsules or lozenges, oral suspensions in dry form and oral supplements in liquid form.

Hesperidin or a derivative according to the invention may moreover be formulated with the usual excipients and components for such oral compositions or food supplements, namely, especially, fatty and/or water-based components, humectants, thickeners, such as magnesium stearate, preserving agents, texture, taste and/or coating agents, antioxidants, preserving agents and colorants that are common in the food sector, other than hesperidin or a derivative thereof.

The formulating agents and excipients for an oral composition, and especially for food supplements, are known in this field and will not be the subject of a detailed description herein. For ingestion, many embodiments of oral compositions and especially of food supplements are possible. They are formulated via the usual processes for producing sugar-coated tablets, gel capsules, gels, controlled-release hydrogels, emulsions, tablets or capsules.

In particular, hesperidin or a derivative thereof according to the invention may be incorporated into any other form of food supplement or of enriched foods, for example food bars, or compacted or non-compacted powders. The powders may be diluted with water, in soda, dairy products or soya derivatives, or may be incorporated into food bars.

According to one particular embodiment, the oral compositions under consideration according to the invention may be formulated in compositions in an encapsulated form so as to significantly improve their shelf life. In such a case, the presence of a capsule may in particular retard or prevent the degradation of the active agents in the gastrointestinal tract.

The cosmetic treatment process of the invention may be performed especially by administering the cosmetic and/or dermatological compositions as defined above according to the usual technique for using these compositions.

The cosmetic process according to the invention may be performed by topical and/or oral, preferably oral, for example daily, administration of cosmetic and/or dermatological compositions according to the invention, which may be formulated, for example, in the form of gels, lotions, emulsions or ingestible supports.

Oral administration consists in ingesting in one or more intakes an oral composition as defined above.

According to one embodiment, the process in accordance with the invention may comprise the administration in two steps of a composition according to the invention, i.e. a first step comprising the administration, over a period of time, of a composition in accordance with the invention orally, followed or preceded, in particular followed, by a second step comprising the administration, over another period of time, of a composition in accordance with the invention topically.

According to one embodiment variant of the invention, the cosmetic process comprises at least one step of oral administration of an effective amount of hesperidin or a derivative thereof, and of an effective amount of hesperidin or a derivative thereof.

The process according to the invention may comprise a single, preferably oral, administration.

According to one embodiment, the administration is repeated, for example two to three times daily or more and generally over a prolonged period of at least 4 weeks, or even from 4 to 15 weeks, with, where appropriate, one or more periods of interruption.

In the description and the examples that follow, unless otherwise indicated, the percentages are weight percentages and the ranges of values given in the form “between . . . and . . . ” include the stated lower and upper limits.

The ingredients are mixed before being put in form, in order and under conditions that are readily determined by a person skilled in the art.

The examples below are presented as non-limiting illustrations of the field of the invention.

### EXAMPLES

### Example 1

Capsule in Accordance with the Invention for Oral Application

| Compounds | mg/capsule |
|---|---|
| Hesperidin | 500 |
| Glycerol | 150 |
| Magnesium stearate | 0.02 |
| Natural flavour | qs |

One to three capsules may be taken each day.

### Example 2

Proteomic Results

The test product consists of hesperidin and is administered orally.

The active agent was tested alone in a randomized double-blind study.

66 women were divided into two groups, one of these groups testing maltodextrin (n=33 group A), the other testing hesperidin (n=33 group B).

The supplementation was 500 mg of hesperidin per day.

The evolution of various cutaneous markers was studied by proteomics on samples of isolated stratum corneum as indicated hereinbelow.

A sample is taken at times D1 and D168 with varnish strips in order to remove only part of the stratum corneum, i.e. at most the first 4 to 5 layers of stratum corneum.

To do this, a 41 μm Nylon filter gauze of Millipore NY41 type is applied to a predefined area of the left leg. Next, a transparent varnish of reference 614254/T.D. comprising: 6.86 g of nitrocellulose, 2.94 g of isopropanol, 7.35 g of hypoallergenic alkyl resin, 7.7 g of acetyl tributyl citrate and 75.15 g of ethyl acetate, is spread on using a brush (15 mm) and then left to dry for 15 minutes.

The Nylon gauze is then recovered using tweezers by pulling off the varnish strip in a single abrupt action.

The varnished strips are stored flat at −20° C. in plastic sachets.

These skin samples (varnish strips of stratum corneum) were then analysed by proteomics by means of an “isobaric marking” technique to evaluate the expression of the protein(s) under study.

This “isobaric marking” technique or iTRAQ is based on the marking of the tryptic peptides with a series of reagents, which are said to be isobaric since they all have a molecular mass of 145 Da, and forming a covalent bond with the primary amines of the amino-terminal end of the side chain of the lysine residues.

The marked peptides are detected by mass spectrometry with the intrinsic mass of the peptide +145 Da, originating from the reagent. In the peptide fragmentation step, the contribution of each of the reagents is assessed by the release of ions (fragments) having different specific masses.

Such a method is described precisely by Zieske (J. Exp. Bot., 2006, 57:1501) or Wiese et al. (Proteomics, 2007, 7:340).

The results of the differential proteomic analysis show that a supplementation with hesperidin significantly stimulates the expression of dermicidin, when compared with the administration of a placebo agent, namely maltodextrin, used in place of hesperidin with a control group (group A indicated previously).

Stimulation of the expression of the abovementioned protein makes it possible to reduce the colonization by the main Corynebacterium microorganisms responsible by their metabolism for the development of body odour.

Such a decrease, which is a consequence of the supplementation with hesperidin, contributes towards re-establishing an equilibrated ecoflora on the epidermis, which has the consequence of reducing the body odour.

1-9. (canceled)

10. Cosmetic method for treating and/or preventing body odour, comprising at least one step of oral administration of hesperidin or a derivative thereof.

11. Method according to the preceding claim, wherein hesperidin or a derivative thereof is administered in an oral composition to an individual.

12. Method according to claim 11, wherein the body odour is associated directly or indirectly with the presence of bodily fluids secreted on an epidermis.

13. Method according to claim 11 for stimulating the expression of dermicidin in epidermal stratum corneum.

14. Method according to claim 11 wherein hesperidin or a derivative thereof is used in the composition in a content of between 0.001% and 30% by weight relative to the total weight of the composition.

15. Method according to claim 11 wherein the daily dose of hesperidin or of a derivative thereof is from 0.5 to 2500 mg per day.

16. Method according to claim 11 wherein the composition also comprises at least one additional active agent for treating and/or preventing body odour, other than hesperidin or a derivative thereof.

17. Method according to claim 16, wherein the additional active agent is present in a content ranging from 0.1% to 10% by weight, relative to the total weight of the composition comprising it.

18. Method according to claim 11, wherein the composition is used in an ingestible support, this support being chosen from milk, yogurt, cheese, fermented milks, milk-based fermented products, ice creams, products based on fermented cereals, milk-based powders, infant and baby formulas, food products of confectionary type, chocolates, cereals, animal feed in particular for pets, tablets, gel capsules or lozenges, oral supplements in dry form and oral supplements in liquid form.
